(19) United States
(12) Patent Application Publication
(10) Pub. No.: US 2015/0314124 A1
(43) Pub. Date: Nov. 5, 2015

(54) SYSTEMS AND METHODS FOR DETECTING AN OCCURRENCE OF A STAPEDIUS REFLEX WITHIN A COCHLEAR IMPLANT PATIENT

(71) Applicant: ADVANCED BIONICS AG, Staefa (CH)

(72) Inventors: Kinuko Masaki, San Francisco, CA (US); Aniket Saoji, Naperville, IL (US)

(21) Appl. No.: 14/648,652

(22) PCT Filed: Dec. 6, 2012
(86) PCT No.: PCT/US12/68214

§ 371 Date: May 29, 2015

(51) Int. Cl. A61N 1/36; H04R 25/00; A61N 1/08

(52) U.S. Cl. CPC A61N 1/36032; A61N 1/08; H04R 25/30

(57) ABSTRACT

An exemplary system includes 1) a probe frequency management facility configured to determine a resonant frequency of a middle ear space of a patient fitted with a cochlear implant system and select, in accordance with the determined resonant frequency, a probe frequency for use by a middle ear analyzer, and 2) a control facility configured to direct the middle ear analyzer to generate a measurement probe tone having the selected probe frequency and to use the measurement probe tone to monitor for an occurrence of a stapedius reflex in response to the cochlear implant system applying electrical stimulation by way of one or more electrodes implanted within the patient. Corresponding systems and methods are also disclosed.

### BACKGROUND INFORMATION

To overcome some types of hearing loss, numerous cochlear implant systems (also known as cochlear prostheses) have been developed. Cochlear implant systems bypass the hair cells in the cochlea by presenting electrical stimulation directly to the auditory nerve fibers by way of one or more channels formed by an array of electrodes implanted in the cochlea. Direct stimulation of the auditory nerve fibers leads to the perception of sound in the brain and at least partial restoration of hearing function.

When a cochlear implant system is initially implanted in a patient, and during follow-up tests and checkups thereafter, it is usually necessary to fit the cochlear implant system to the patient. Such “fitting” includes adjustment of the base amplitude or intensity of the various stimuli generated by the cochlear implant system from the factory settings (or default values) to values that are most effective and comfortable for the patient. For example, the intensity or amplitude and/or duration of the individual stimulation pulses provided by the cochlear implant system may be mapped to an appropriate dynamic audio range so that the appropriate “loudness” of sensed audio signals is perceived. That is, loud sounds should be sensed by the patient at a level that is perceived as loud, but not painfully loud. Soft sounds should similarly be sensed by the patient at a level that is soft, but not so soft that the sounds are not perceived at all.

Hence, fitting and adjusting the intensity of the stimuli and other parameters of a cochlear implant system to meet a particular patient's needs requires the determination of one or more most comfortable current levels (“M levels”). An M level refers to a stimulation current level applied by a cochlear implant system at which the patient is most comfortable. M levels typically vary from patient to patient and from channel to channel in a multichannel cochlear implant.

M levels are typically determined based on subjective feedback provided by cochlear implant patients. For example, a clinician may present various stimuli to a patient and then analyze subjective feedback provided by the patient as to how the stimuli were perceived. Such subjective feedback typically takes the form of either verbal (adult) or non-verbal (child) feedback. Unfortunately, relying on subjective feedback in this manner is difficult, particularly for those patients who may have never heard sound before and/or who have never heard electrically-generated “sound.” For young children, the problem is exacerbated by a short attention span, as well as difficulty in understanding instructions and concepts, such as high and low pitch, softer and louder, same and different. Moreover, many patients, such as infants and those with multiple disabilities, are completely unable to provide subjective feedback.

Hence, it is often desirable to employ an objective method of determining M levels for a cochlear implant patient. One such objective method involves applying electrical stimulation with a cochlear implant system to a patient until a stapedius reflex (i.e., an involuntary muscle contraction that occurs in the middle ear in response to acoustic and/or electrical stimulation) is elicited. This is because the current level required to elicit a stapedius reflex within a patient (referred to herein as a “stapedius reflex threshold”) is highly correlated with (e.g., in many cases, substantially equal to) an M level corresponding to the patient. However, currently available techniques for measuring the current level at which a stapedius reflex actually occurs within a cochlear implant patient are unreliable, time consuming, and difficult to implement (especially with pediatric patients).

For example, a middle ear analyzer is often used to objectively measure a sound level at which an acoustic stimulus elicits a stapedius reflex in a non-cochlear implant patient by applying the acoustic stimulus to the ear of the non-cochlear implant patient and recording the resulting change in acoustic immittance. It would be desirable for a middle ear analyzer to be adapted for a cochlear implant patient by configuring the middle ear analyzer to record a change in acoustic immittance that occurs in response to electrical stimulation provided by the cochlear implant system. The change in the acoustic immittance could then be used to derive the stapedius reflex threshold.

However, it is currently very difficult to detect an occurrence of a stapedius reflex in an ear that is fitted with a cochlear implant system. The exact reasons for this are unknown. This limitation may be overcome in unilateral cochlear implant patients (i.e., patients with a cochlear implant in only one ear) by detecting an occurrence of a stapedius reflex in the non-implanted ear. Unfortunately, no such workaround exists for bilateral cochlear implant patients (i.e., patients with a cochlear implant in both ears).

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary configuration 100 that may be used to elicit and measure a stapedius reflex according to principles described herein.

FIG. 2 shows various components of a middle ear analyzer according to principles described herein.

FIG. 3 shows various components of an interface system according to principles described herein.

FIG. 4 shows various components of a cochlear implant system according to principles described herein.

FIG. 5 illustrates an exemplary implementation of the configuration of FIG. 1 according to principles described herein.

FIG. 6 illustrates another exemplary implementation of the configuration of FIG. 1 according to principles described herein.

FIG. 7 illustrates an exemplary stapedius reflex elicitation and measurement system according to principles described herein.

FIG. 8 illustrates an exemplary method of detecting an occurrence of a stapedius reflex within a cochlear implant patient according to principles described herein.

FIG. 9 illustrates an exemplary computing device according to principles described herein.

### DETAILED DESCRIPTION

Systems and methods for detecting an occurrence of a stapedius reflex within a cochlear implant patient are described herein. For example, an exemplary system may 1) determine a resonant frequency of a middle ear space of a patient fitted with a cochlear implant system, 2) select, in accordance with the determined resonant frequency, a probe frequency for use by a middle ear analyzer, 3) direct the middle ear analyzer to generate a measurement probe tone having the selected probe frequency, and 4) direct the middle ear analyzer to use the measurement probe tone to monitor for an occurrence of a stapedius reflex in response to the cochlear implant system applying electrical stimulation by way of one or more electrodes implanted within the patient.

It has been discovered that by using the resonant frequency of the middle ear space of the cochlear implant patient (or simply “the resonant frequency”) or a frequency relatively close to the resonant frequency as the probe frequency of the measurement probe tone used by the middle ear analyzer to monitor for a stapedius reflex, the likelihood of detecting the stapedius reflex is increased compared to conventional techniques that use a much lower frequency as the probe frequency.

To illustrate, a conventional technique that is used to monitor for an occurrence of a stapedius reflex in a patient involves using a middle ear analyzer to generate and present a measurement probe tone to an ear of the patient. The middle ear analyzer may measure a signal that reflects off the tympanic membrane in response to the measurement probe tone and accordingly derive an acoustic immittance of the middle ear space. When a stimulus (either acoustic or electric) is applied to either ear of the patient, the tympanic membrane may stiffen, which may result in the middle ear analyzer detecting a change in the signal reflecting off the tympanic membrane (and therefore a change in the acoustic immittance). A change in acoustic immittance that is greater than a predetermined threshold may be indicative of an occurrence of a stapedius reflex.

The frequency of the measurement probe tone is conventionally set to be relatively low (e.g., 226 Hz). At this frequency, it is often difficult or even impossible to detect an occurrence of a stapedius reflex in the same ear that has a cochlear implant implanted therein (referred to herein as an “implanted ear”). However, it has been discovered that by increasing the frequency of the measurement probe tone to be relatively close or equivalent to the resonant frequency of the patient (which is patient-specific and typically between 800 and 1200 Hz), the likelihood of detecting an occurrence of a stapedius reflex in an implanted ear significantly increases. Likewise, the likelihood of detecting an occurrence of a stapedius reflex in a non-implanted ear (i.e., an ear that does not have a cochlear implant implanted therein) also increases.

The systems and methods described herein may increase the efficiency and safety of detecting an occurrence of a stapedius reflex within a cochlear implant patient. For example, the tympanic membrane is the most sensitive at the resonant frequency. Hence, smaller changes in the tympanic membrane may be more easily detected using a probe frequency that is close or equal to the resonant frequency. This may reduce the time it takes for a stapedius reflex to be detected in a patient, which may be especially beneficial for certain types of patients (e.g., pediatric patients) with short attention spans. This may also increase patient safety by preventing the patient from being over-stimulated during a fitting session.

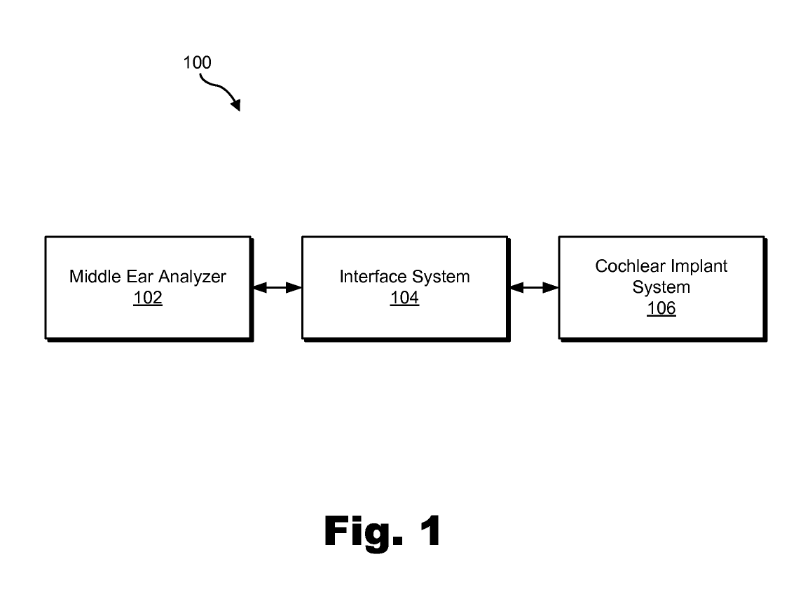

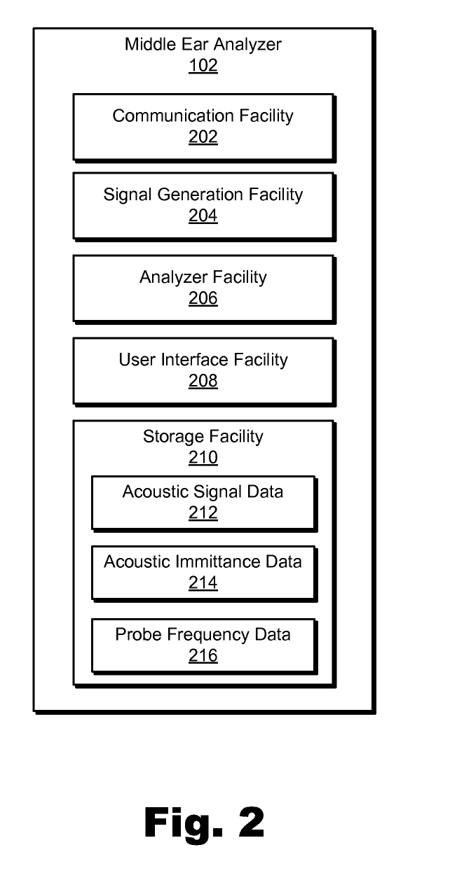

FIG. 1 illustrates an exemplary configuration 100 that may be used to elicit and measure a stapedius reflex. Configuration 100 may be configured to elicit one or more stapedius reflexes within a cochlear implant patient and identify one or more current levels at which the one or more stapedius reflexes occur (i.e., one or more stapedius reflex thresholds). To this end, configuration 100 may include a middle ear analyzer 102, an interface system 104, and a cochlear implant system 106 communicatively coupled to one another. Each of these components will now be described in connection with FIGS. 2-4.

FIG. 2 shows various components of middle ear analyzer 102. As shown, middle ear analyzer 102 may include, without limitation, a communication facility 202, a signal generation facility 204, an analyzer facility 206, a user interface facility 208, and a storage facility 210 communicatively coupled to one another. It will be recognized that although facilities 202-210 are shown to be separate facilities in FIG. 2, any of facilities 202-210 may be combined into fewer facilities, such as into a single facility, or divided into more facilities as may serve a particular implementation.

Communication facility 202 may be configured to facilitate communication between middle ear analyzer 102 and interface system 104 (e.g., by way of a probe). Communication facility 202 may additionally or alternatively be configured to facilitate data transmission between middle ear analyzer 102 and interface system 104. For example, communication facility 202 may be configured to facilitate transmission of an acoustic signal to interface system 104.

Signal generation facility 204 may be configured to generate an acoustic signal used (e.g., by interface system 104) to direct cochlear implant system 106 to apply electrical stimulation having a current level based on a sound level of the acoustic signal by way of one or more electrodes implanted within a patient. As will be described in more detail below, signal generation facility 204 may incrementally increase the sound level of the acoustic signal until the occurrence of the stapedius reflex is detected (e.g., by analyzer facility 206).

Analyzer facility 206 may be configured to perform one or more analysis functions. For example, analyzer facility 206 may be configured to generate a measurement probe tone having a probe frequency selected in accordance with a resonant frequency of a middle ear space of the patient and use the measurement probe tone to monitor for an occurrence of a stapedius reflex in response to cochlear implant system 106 applying the electrical stimulation by way of the one or more electrodes. Exemplary manners in which the resonant frequency may be determined and used to select the probe frequency will be described in more detail below.

Analyzer facility 206 may use the measurement probe tone to monitor for an occurrence of a stapedius reflex in any suitable manner. For example, analyzer facility 206 may use the measurement probe tone to measure and record a change in acoustic immittance that occurs in response to the electrical stimulation applied by cochlear implant system 106. As used herein, “acoustic immittance” may refer to an acoustic impedance, admittance, and/or combination thereof. For example, acoustic immittance may refer to a ratio of sound pressure to volume velocity within the ear canal that occurs in response to application of electrical and/or acoustic stimulation of the auditory pathway of the patient.

In some examples, signal generation facility 204 may be configured to incrementally increase the sound level of the acoustic signal provided to cochlear implant system 106 by way of interface system 104 until analyzer facility 206 (or a clinician) detects a change in the acoustic immittance that indicates an occurrence of a stapedius reflex. This may be performed in any suitable manner. For example, during a particular stapedius reflex measurement session, signal generation facility 204 may incrementally increase the sound level of the acoustic signal (e.g., step through a sequence of discrete sound levels specified in a mapping data set maintained by interface system 104) until analyzer facility 206 detects that the change in acoustic immittance reaches a predetermined threshold. In some examples, signal generation facility 204 may be configured to cease providing transmitting the acoustic signal once analyzer facility 206 determines that a stapedius reflex has occurred.

In some examples, signal generation facility 204 may be configured to stop increasing the sound level of the acoustic signal once the sound level is equal to a predetermined maximum threshold level (e.g., an “uncomfortable level” or “U level” of a cochlear implant patient) even if a stapedius reflex has not been detected. The U level of a cochlear implant patient may be determined in any suitable manner. In this manner, the patient will not be over-stimulated.

In some examples, signal generation facility 204 may be configured to set a frequency of the acoustic signal that is transmitted to interface system 104 in order to specify a set of one or more electrodes by which cochlear implant system 106 is to apply electrical stimulation. For example, a first frequency may designate a first set of one or more electrodes (e.g., electrodes one through four in an electrode array), a second frequency may designate a second set of one or more electrodes (e.g., electrodes five through eight in an electrode array), etc. It will be recognized that any combination of electrodes (e.g., all of the electrodes included in the electrode array) may be specified by the frequency of the acoustic signal provided by signal generation facility 204.

User interface facility 208 may be configured to provide one or more graphical user interfaces (“GUIs”) associated with an operation of middle ear analyzer 102. For example, a GUI may be provided and configured to facilitate user input identifying various frequencies and sound levels that the clinician desires to test with a particular patient.

Storage facility 210 may be configured to maintain acoustic signal data 212 representative of one or more acoustic signals generated by signal generation facility 204, acoustic immittance data 214 representative of one or more acoustic immittance measurements made by analyzer facility 206, and/or probe frequency data 216 representative of or otherwise associated with a probe frequency of the measurement probe signal used by analyzer facility 206 to monitor for an occurrence of a stapedius reflex within the patient. It will be recognized that storage facility 210 may maintain additional or alternative data as may serve a particular implementation.

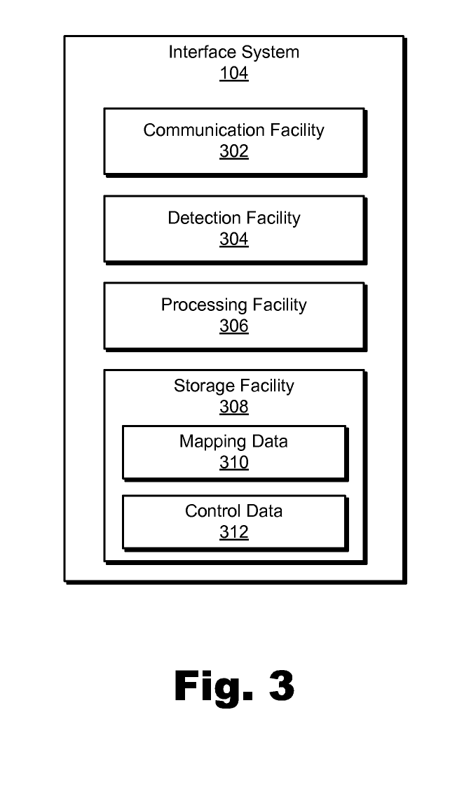

FIG. 3 shows various components of interface system 104. As shown, interface system 104 may include, without limitation, a communication facility 302, a detection facility 304, a processing facility 306, and a storage facility 308 communicatively coupled to one another. It will be recognized that although facilities 302-308 are shown to be separate facilities in FIG. 3, any of facilities 302-308 may be combined into fewer facilities, such as into a single facility, or divided into more facilities as may serve a particular implementation.

Communication facility 302 may be configured to facilitate communication between interface system 104 and middle ear analyzer 102. Communication facility 302 may be further configured to facilitate communication between interface system 104 and cochlear implant system 106. To this end, communication facility 302 may be configured to employ any suitable combination of ports, communication protocols, and data transmission means.

Detection facility 304 may be configured to receive one or more acoustic signals transmitted by a middle ear analyzer 102 and detect a sound level and frequency of the one or more acoustic signals. Detection facility 304 may employ any suitable signal processing heuristic to detect the sound level and frequency of an acoustic signal as may serve a particular implementation.

Processing facility 306 may be configured to perform any suitable processing operation related to one or more acoustic signals detected by detection 304. For example, processing facility 306 may be configured to manage (e.g., maintain, generate, update, etc.) mapping data representative of an association between a plurality of sound levels and a plurality of current levels and between a plurality of frequencies and a plurality of electrodes. Mapping data may be maintained in the form of a look-up table, in a database, and/or in any other manner as may serve a particular implementation.

To illustrate, Table 1 illustrates a mapping data set representative of an exemplary association between a plurality of sound levels and a plurality of current levels that may be maintained by processing facility 306.

TABLE 1

| Sound Level | Current Level |
|---|---|
| (dB SPL) | (CU) |
| 80 | 110 |
| 85 | 120 |
| 90 | 130 |
| 95 | 140 |
| 100 | 150 |

As shown in Table 1, the mapping data set indicates that a sound level of 80 dB SPL is mapped to a current level of 110 clinical units (“CU”), a sound level of 85 dB SPL is mapped to a current level of 120 CU, a sound level of 90 dB SPL is mapped to a current level of 130 CU, a sound level of 95 dB SPL is mapped to a current level of 140 CU, and a sound level of 100 dB SPL is mapped to a current level of 150 CU. As will be described below, processing facility 306 may use a mapping data set similar to that illustrated in Table 1 to identify a current level that is associated with a sound level of a particular acoustic signal detected by detection facility 304. It will be recognized that the mapping data set illustrated in Table 1 is merely illustrative of the many different mapping data sets that may be utilized and/or generated in accordance with the systems and methods described herein. In some examples, processing facility 306 may interpolate between the various data points included in Table 1 to determine the relationship between sound level and current level for values not specifically included in Table 1. For example, processing facility 306 may use one or more interpolation techniques to determine a current level that corresponds to a sound level of 82 dB SPL, even though this particular sound level is not specifically included in Table 1. Moreover, it will be recognized in some alternative embodiments, an equation may be used to define the relationship between sound level and current level.

In some examples, processing facility 306 may maintain a default mapping data set representative of an association between a plurality of sound levels and a plurality of current levels. The default mapping data set may be used during an initial stapedius reflex measurement session, for example, to identify a current level at which a stapedius reflex occurs (i.e., a stapedius reflex threshold) when electrical stimulation is presented by cochlear implant system 106 by way of a particular set of one or more electrodes. In some examples, processing facility 306 may use the identified current level to generate a refined mapping data set for use during one or more subsequent stapedius reflex measurement sessions.

Table 2 illustrates an exemplary mapping data set representative of an exemplary association between a plurality of frequencies and a plurality of electrodes (e.g., a plurality of electrodes included in an array of electrodes configured to be implanted within a cochlea of a patient) that may be maintained by processing facility 306.

TABLE 2

| Frequency | Electrode |
|---|---|
| (kHz) | Numbers |
| 1 | 1-4 |
| 2 | 5-8 |
| 3 | 9-12 |
| 4 | 13-16 |
| 5 | 1-16 |

As shown in Table 2, the additional mapping data indicates that a frequency of 1 kHz is mapped to electrodes 1 through 4, a frequency of 2 kHz is mapped to electrodes 5 through 8, a frequency of 3 kHz is mapped to electrodes 9 through 12, a frequency of 4 kHz is mapped to electrodes 13 through 16, and a frequency of 5 kHz is mapped to electrodes 1 through 16. Other combinations of electrodes may be represented by other frequencies as may serve a particular implementation. As will be described below, processing facility 306 may use the mapping data set illustrated in Table 2 to identify one or more electrodes that are associated with a frequency of a particular acoustic signal detected by detection facility 304. It will be recognized that the mapping associations between frequency and electrode numbers illustrated in Table 2 are merely illustrative of the many different mapping associations that may be utilized in accordance with the systems and methods described herein.

Processing facility 306 may be further configured to facilitate elicitation and measurement of a stapedius reflex during a stapedius reflex measurement session. For example, an acoustic signal may be transmitted to interface system 104 by middle ear analyzer 102 during a particular stapedius reflex measurement session. Processing facility 306 may utilize the mapping data described above to identify a current level that corresponds to the sound level of the acoustic signal and a set of one or more electrodes that corresponds to the frequency of the acoustic signal. Processing facility 306 may then direct cochlear implant system 106 to apply electrical stimulation having the identified current level by way of the identified set of one or more electrodes.

Processing facility 306 may be further configured to synchronize the middle ear analyzer with the cochlear implant system during a stapedius reflex measurement session. In other words, processing facility 306 may ensure that the current level of the electrical stimulation being provided by cochlear implant system 106 is correlated with the sound level of the acoustic signal as the sound level of the acoustic signal is incrementally increased during the stapedius reflex measurement session.

In some examples, processing facility 306 may synchronize middle ear analyzer 102 and cochlear implant system 106 during the stapedius reflex measurement session in accordance with a mapping data set (e.g., the mapping data set illustrated in Table 1). For example, processing facility 306 may synchronize middle ear analyzer 102 and cochlear implant system 106 during the stapedius reflex measurement session by dynamically translating the sound level of the acoustic signal into a series of increasing current level values in accordance with a mapping data set as the sound level incrementally increases during the stapedius reflex measurement session and directing cochlear implant system 106 to dynamically increase the current level of the electrical stimulation being applied by way of the set of one or more electrodes in accordance with the series of increasing current level values (e.g., by transmitting one or more control parameters to cochlear implant system 106). To illustrate, middle ear analyzer 102 may incrementally step through the various sound levels included in Table 1 until a stapedius reflex is elicited. As middle ear analyzer 102 incrementally steps through the various sound levels, processing facility 306 may direct cochlear implant system 106 to incrementally increase the current level of the electrical stimulation being applied by cochlear implant system 106 in accordance with the current level values included in Table 1.

An exemplary manner in which processing facility 306 synchronizes middle ear analyzer 102 and cochlear implant system 106 is more fully described in co-pending PCT Application No. PCT/US12/67354, entitled “Systems and Methods for Facilitating Use of a Middle Ear Analyzer in Determining One or More Stapedius Reflex Thresholds Associated with a Cochlear Implant Patient,” filed Nov. 30, 2012, and incorporated herein by reference in its entirety.

Once a stapedius reflex has been detected by middle ear analyzer 102, processing facility 306 may identify a current level of the electrical stimulation at which the stapedius reflex occurs. This may be performed in any suitable manner. For example, processing facility 306 may identify the current level by identifying the last current level used before the stapedius reflex measurement session is terminated. In some examples, processing facility 306 may designate the identified current level as a stapedius reflex threshold associated with the set of one or more electrodes by which electrical stimulation is being applied during the stapedius reflex measurement session.

In some examples, processing facility 306 may be configured to prevent cochlear implant system 106 from increasing the current level of the electrical stimulation applied to the patient beyond a U level associated with the patient. As mentioned, the U level represents an “uncomfortable level” associated with the patient. Stimulation above the U level may result in discomfort, pain, and/or damage to the patient. Hence, limiting cochlear implant system 106 from increasing the current level beyond the U level of a patient may ensure patient comfort and safety.

Processing facility 306 may be further configured to present one or more GUIs and receive user input by way of the one or more GUIs. For example, processing facility 306 may be configured to detect an occurrence of a stapedius reflex and designate the current level associated with the stapedius reflex as being an M level associated with the patient. The detection of the occurrence of the stapedius reflex may be performed automatically by processing facility 306 or in response to user input provided by way of one or more GUIs presented by processing facility 306. For example, processing facility 306 may receive user input representative of a sound level at which a stapedius reflex occurred during the application of electrical stimulation by cochlear implant system 106. Based on the user input and on the mapping data, processing facility 306 may determine a current level at which the stapedius reflex occurred, designate the current level as an M level associated with the patient, and present data representative of the M level within a GUI.

Processing facility 306 may be further configured to perform one or more calibration operations associated with a particular middle ear analyzer. For example, interface system 104 may be used in connection with a variety of different middle ear analyzers. Each middle ear analyzer may be calibrated upon being connected to interface system 104 so that appropriate current levels are applied to the patient.

In some alternative examples, it may be desirable for a user of interface system 104 to specify a particular group of electrodes to be tested (i.e., a group of electrodes for which a stapedius reflex threshold is to be determined). For example, a clinician may desire to determine the M level for a single electrode. To this end, processing facility 306 may provide a GUI configured to facilitate identification by a user of one or more specific electrodes. In response to receiving this user input, processing facility 306 may direct middle ear analyzer 102 to provide an acoustic signal having a frequency associated with the identified one or more electrodes. Detection facility 304 may detect the sound level of an acoustic signal, and processing facility 306 may identify a current level associated with the sound level based on the mapping data. Processing facility 306 may then direct a cochlear implant system to apply electrical stimulation having the identified current level by way of the identified one or more electrodes.

Storage facility 308 may be configured to maintain mapping data 310 (e.g., one or more mapping data sets) managed by processing facility 306 and control data 312 (e.g., one or more control parameters) generated by processing facility 306. It will be recognized that storage facility 308 may maintain additional or alternative data as may serve a particular implementation.

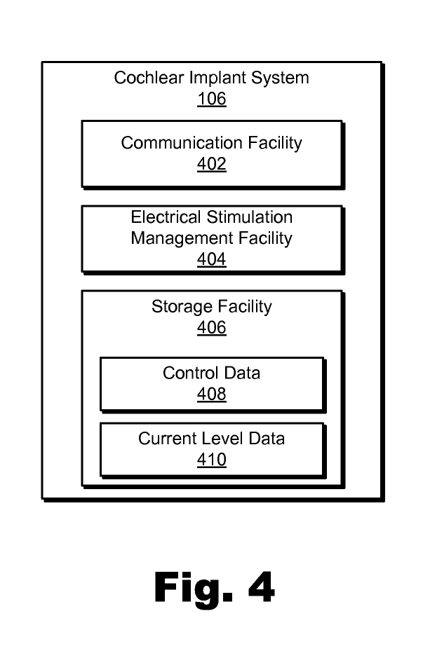

FIG. 4 shows various components of cochlear implant system 106. As shown, cochlear implant system 106 may include, without limitation, a communication facility 402, an electrical stimulation management facility 404, and a storage facility 406 communicatively coupled to one another. It will be recognized that although facilities 402-406 are shown to be separate facilities in FIG. 4, any of facilities 402-406 may be combined into fewer facilities, such as into a single facility, or divided into more facilities as may serve a particular implementation.

Communication facility 402 may be configured to facilitate communication between cochlear implant system 106 and interface system 104. To this end, communication facility 402 may be configured to employ any suitable combination of ports, communication protocols (e.g., wired and/or wireless communication protocols), and data transmission means.

Electrical stimulation management facility 404 may be configured to perform any suitable electrical stimulation operation as may serve a particular implementation. For example, electrical stimulation management facility 404 may receive control data representative of a particular current level and one or more electrodes from interface system 104. Based on this control data, electrical stimulation management facility 404 may generate and apply electrical stimulation having the particular current level to one or more stimulation sites within a cochlear implant patient by way of the one or more electrodes. The electrical stimulation may be generated and applied in any suitable manner as may serve a particular implementation. For example, a sound processor located external to the patient may use the control data to generate one or more stimulation parameters configured to direct a cochlear implant implanted within the patient to generate and apply the electrical stimulation.

In some examples, electrical stimulation management facility 404 may incrementally increase the current level of the electrical stimulation in response to middle ear analyzer 102 incrementally increasing a sound level of an acoustic signal until a stapedius reflex that occurs in response to the electrical stimulation is detected. Once a stapedius reflex occurs, electrical stimulation management facility 404 may identify the current level at which the stapedius reflex occurs and direct storage facility 406 to store data representative of the current level. Electrical stimulation management facility 404 may then utilize the stored data to determine one or more M levels associated with the set of one or more electrodes for use in one or more stimulation programs (i.e., one or more stimulation programs used by cochlear implant system 106 during a normal operation subsequent to a fitting session in which cochlear implant system 106 is coupled to interface system 104). For example, electrical stimulation management facility 404 may use the stored data to generate and apply electrical stimulation having a current level substantially equal to the determined M levels. Electrical stimulation management facility 404 may then generate one or more user-selectable stimulation programs that utilize the generated M levels.

Storage facility 406 may be configured to maintain control data 408 received from interface system 104 and current level data 410 representative of one or more current levels at which one or more stapedius reflexes occur. It will be recognized that storage facility 406 may maintain additional or alternative data as may serve a particular implementation.

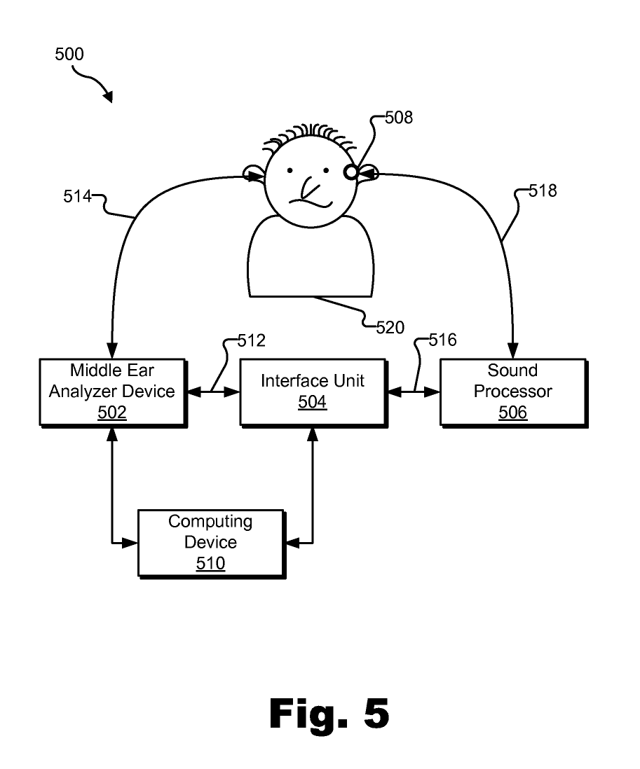

FIG. 5 illustrates an exemplary implementation 500 of configuration 100. As shown, implementation 500 may include a middle ear analyzer device 502, an interface unit 504, a sound processor 506, a cochlear implant 508, and a computing device 510. Implementation 500 may further include a stimulation probe 512 configured to communicatively couple middle ear analyzer device 502 and interface unit 504 and a detection probe 514 configured to be coupled to middle ear analyzer device 502 and detect a change in immittance that occurs as a result of electrical stimulation applied by way of one or more electrodes (not shown) communicatively coupled to cochlear implant 508.

Middle ear analyzer 102, interface system 104, and cochlear implant system 106 may each be implemented by one or more components illustrated in FIG. 5. For example, middle ear analyzer 102 may be implemented by middle ear analyzer device 502, stimulation probe 512, detection probe 514, and computing device 510. Interface system 104 may be implemented by interface unit 504 and computing device 510. Cochlear implant system 106 may be implemented by sound processor 506 and cochlear implant 508.

Each of the components shown in FIG. 5 will now be described in more detail. Middle ear analyzer device 502 may include any suitable middle ear analyzer (e.g. an off-the-shelf middle ear analyzer) configured to perform one or more of the middle ear analyzer operations described herein. For example, in the configuration of FIG. 5, middle ear analyzer device 502 may operate in a contralateral stimulation mode in which middle ear analyzer device 502 is configured to direct sound processor 506 and cochlear implant 508 to apply electrical stimulation to a first ear of the patient (e.g., by providing one or more acoustic signals with stimulation probe 512 to interface unit 504, which in turn directs sound processor 506 and cochlear implant 508 to apply the electrical stimulation based on the one or more acoustic signals) and record a resulting change in immittance in a second ear of the patient using detection probe 514. Alternatively, as will be described below, middle ear analyzer device 502 may operate in an ipsilateral stimulation mode in which the stimulation and recording are performed with respect to the same ear.

Interface unit 504 may be configured to perform one or more interface operations as described herein. For example, interface unit 504 may include any combination of signal receivers, signal transmitters, processors, and/or computing devices configured to receive an acoustic signal transmitted by a middle ear analyzer device 502 by way of stimulation probe 512, detect a sound level and frequency of the acoustic signal, and transmit control data representative of a current level associated with the sound level and one or more electrodes associated with the frequency to sound processor 506.

Interface unit 504 may be coupled directly to middle ear analyzer device 502 by way of stimulation probe 512. Interface unit 504 may also be coupled to sound processor 506 by way of communication channel 516, which may include any suitable wired and/or wireless communication channel as may serve a particular implementation.

Sound processor 506 may include any type of sound processor used in a cochlear implant system as may serve a particular implementation. For example, sound processor 506 may include a behind-the-ear (“BTE”) sound processing unit, a portable speech processor (“PSP”), and/or a body-worn processor.

Cochlear implant 508 may include any suitable auditory prosthesis configured to be at least partially implanted within a patient as may serve a particular implementation. For example, cochlear implant 508 may include an implantable cochlear stimulator, a brainstem implant and/or any other type of auditory prosthesis. In some examples, cochlear implant 508 may be communicatively coupled to a lead having a plurality of electrodes (e.g., sixteen electrodes) disposed thereon. The lead may be configured to be implanted within the patient such that the electrodes are in communication with stimulation sites (e.g., locations within the cochlea) within the patient. As used herein, the term “in communication with” refers to an electrode being adjacent to, in the general vicinity of, in close proximity to, directly next to, or directly on a stimulation site.

Sound processor 506 and cochlear implant 508 may communicate by way of communication channel 518. Communication channel 518 may be wired or wireless as may serve a particular implementation.

Computing device 510 may include any combination of computing devices (e.g., personal computers, mobile computing devices (e.g., mobile phones, tablet computers, laptop computers, etc.), fitting stations, etc.). As shown, computing device 510 may be communicatively coupled (e.g., with one or more cables) to both the middle ear analyzer device 502 and the interface unit 504. As such, computing device 510 may be configured to perform one or more of the operations associated with the middle ear analyzer device 502 and the interface unit 504. For example, computing device 510 may generate and present one or more GUIs by way of a display device (e.g., a display screen included within computing device 510 and/or communicatively coupled to computing device 510) associated with an operation of middle ear analyzer device 502 and/or interface unit 504.

Additionally or alternatively, computing device 510 may be configured to store, maintain, process, and/or otherwise maintain the mapping data utilized by interface system 104. For example, computing device 510 may be configured to maintain a database comprising the mapping data and identify current levels and/or electrodes associated with an acoustic signal received by interface unit 504.

In some alternative examples, separate computing devices may be associated with middle ear analyzer device 502 and interface unit 504. For example, a first computing device may be communicatively coupled to middle ear analyzer device 502 and configured to perform one or more operations associated with middle ear analyzer device 502 and a second computing device may be communicatively coupled to interface unit 504 and configured to perform one or more operations associated with interface unit 504.

In yet another alternative example, interface unit 504 may not be coupled to computing device 510 or to any other computing device. In this example, interface unit 504 may be configured to perform all of the operations associated with interface system 104 as described herein.

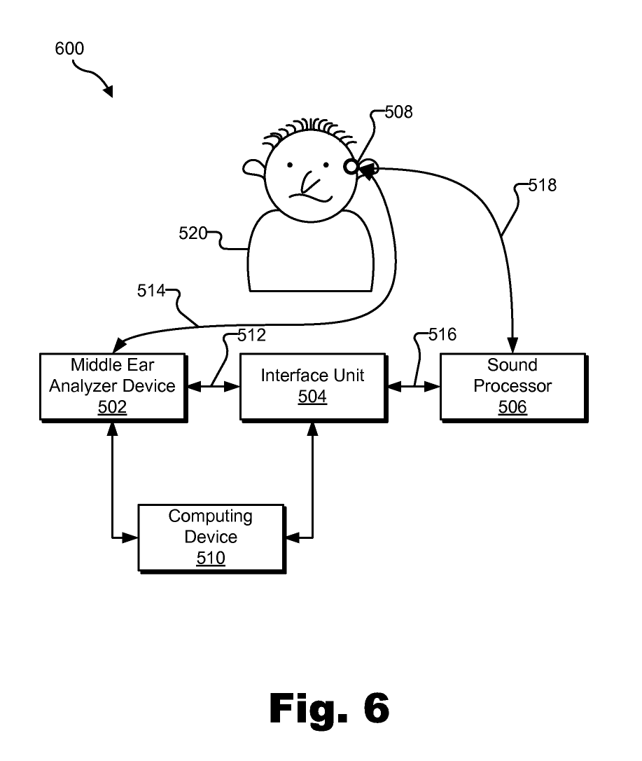

In an exemplary configuration, detection probe 514 is placed within one of the ears of a patient 520. In some examples, as shown in FIG. 5, the ear in which detection probe 514 is placed is contralateral to the ear associated with cochlear implant 508. Alternatively, as shown in the exemplary configuration 600 of FIG. 6, detection probe 514 may be placed within the same (i.e., ipsilateral) ear associated with cochlear implant 508. In this manner, a stapedius reflex may be detected in the same ear to which the electrical stimulation is applied.

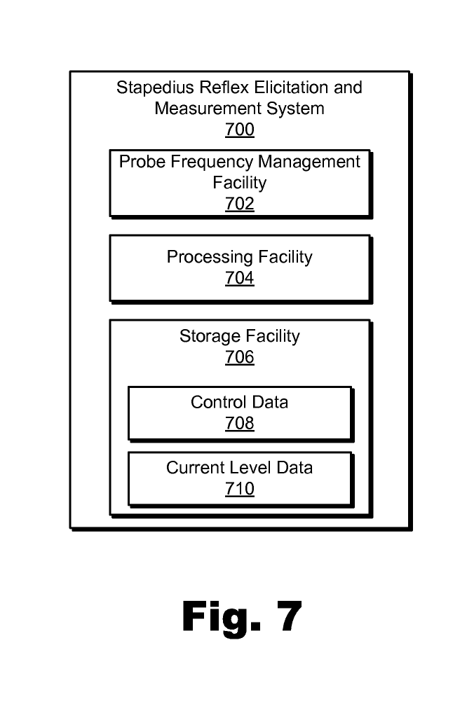

FIG. 7 illustrates an exemplary stapedius reflex elicitation and measurement system 700 (or simply “system 700”) that may be used to perform the various stapedius reflex elicitation and measurement operations described herein. System 700 may be implemented by middle ear analyzer 102, interface system 104, cochlear implant system 106, and/or any implementation thereof. For example, system 700 may be implemented entirely by middle ear analyzer 102 or entirely by interface system 104.

As shown, system 700 may include, without limitation, a probe frequency management facility 702, a control facility 704, and a storage facility 706 selectively and communicatively coupled to one another. It will be recognized that although facilities 702-706 are shown to be separate facilities in FIG. 7, any of facilities 702-706 may be combined into fewer facilities, such as into a single facility, or divided into more facilities as may serve a particular implementation.

Probe frequency management facility 702 may be configured to perform one or more probe frequency management operations as may serve a particular implementation. For example, probe frequency management facility 702 may determine a resonant frequency of a middle ear space of a patient fitted with a cochlear implant system (e.g., cochlear implant system 106). This may be performed in any suitable manner.

For example, probe frequency management facility 702 may determine the resonant frequency by directing the middle ear analyzer to perform a multiple-frequency tympanometry procedure with respect to the patient. In multiple-frequency tympanometry, the middle ear analyzer measures the tympanogram as it sweeps through a series of frequencies (e.g., 250 to 2000 Hz). The resonant frequency may be derived from the resultant tympanogram using any suitable technique known in the art.

Additionally or alternatively, probe frequency management facility 702 may determine the resonant frequency in response to input provided by a user (e.g., a clinician). For example, a clinician may manually input data representative of the resonant frequency of a patient (e.g., by way of a GUI provided by probe frequency management facility 702). Probe frequency management facility 702 may determine the resonant frequency in accordance with the manually input data.

Additionally or alternatively, probe frequency management facility 702 may determine the resonant frequency in accordance with a user profile associated with the patient. For example, data representative of a user profile associated with a patient may be maintained by middle ear analyzer 102, interface system 104, and/or cochlear implant system 106. The user profile may include any suitable information descriptive of or otherwise related to the patient. In some examples, the user profile may specify a resonant frequency associated with a middle ear space of the patient. Alternatively, probe frequency management facility 702 may estimate the resonant frequency of the patient based on the information contained within the user profile of the patient. For example, the user profile may specify a gender, age, and/or any other trait of the patient. Based on this information, probe frequency management facility 702 may estimate that the resonant frequency falls within a certain range of frequencies.

Probe frequency management facility 702 may be further configured to select a probe frequency for use by a middle ear analyzer (e.g., middle ear analyzer 102) in accordance with the determined resonant frequency. For example, probe frequency management facility 702 may select the probe frequency by designating the determined resonant frequency itself as the probe frequency. This may be beneficial in cases where the middle ear analyzer is capable of using an exact frequency specified by a user as the probe frequency.

However, in some middle ear analyzer configurations, the middle ear analyzer may only use one of a predetermined number of preset frequency options as the probe frequency. For example, some middle ear analyzers have three preset frequency options (e.g., 226 Hz, 676 Hz, and 1000 Hz) that may be used as the probe frequency.

Hence, in these cases, probe frequency management facility 702 may select the probe frequency in accordance with the determined resonant frequency by selecting a preset frequency option that most closely matches the determined resonant frequency. To illustrate, a particular middle ear analyzer may have three preset frequency options of 226 Hz, 676 Hz, and 1000 Hz. The determined resonant frequency associated with the patient may be 1100 Hz. Hence, probe frequency management facility 702 may select the 1000 Hz frequency option as the probe frequency. It will be recognized that the selection of a preset frequency option may be constrained by one or more rules (e.g., that the selected preset frequency option must be less than the determined resonant frequency).

Control facility 704 may be configured to direct the middle ear analyzer to generate a measurement probe tone having the selected probe frequency and to use the measurement probe tone to monitor for an occurrence of a stapedius reflex in response to the cochlear implant system applying electrical stimulation by way of one or more electrodes implanted within the patient. This may be performed in any of the ways described herein. For example, control facility 704 may direct the middle ear analyzer to use the measurement probe tone to monitor for the occurrence of the stapedius reflex by directing the middle ear analyzer to use the measurement probe tone to record a change in acoustic immittance that occurs in response to the cochlear implant system applying the electrical stimulation.

In some examples, control facility 704 may direct the middle ear analyzer to apply the measurement probe tone to an implanted ear (e.g., by way of detection probe 514). Additionally or alternatively, control facility 704 may direct the middle ear analyzer to apply the measurement probe tone to a non-implanted ear. In this manner, the systems and methods described herein may be used to monitor for an occurrence of a stapedius reflex in either ear.

Control facility 704 may be further configured to identify a current level at which a stapedius reflex occurs (i.e., a stapedius reflex threshold) and use the identified current level to determine one or more most comfortable stimulation levels (“M levels”) associated with the one or more electrodes by which the electrical stimulation is applied by the cochlear implant system. This may be performed in any of the ways described herein.

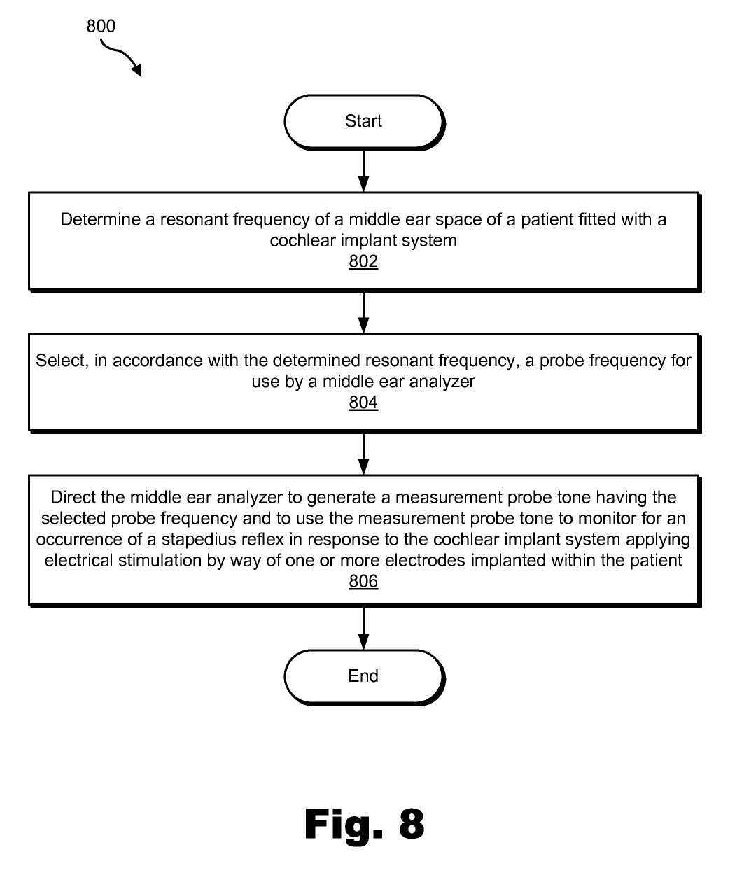

FIG. 8 illustrates an exemplary method 800 of detecting an occurrence of a stapedius reflex within a cochlear implant patient. While FIG. 8 illustrates exemplary steps according to one embodiment, other embodiments may omit, add to, reorder, and/or modify any of the steps shown in FIG. 8. One or more of the steps shown in FIG. 8 may be performed by stapedius reflex elicitation and measurement system 700 and/or any implementation thereof.

In step 802, a stapedius reflex elicitation and measurement system determines a resonant frequency of a middle ear space of a patient fitted with a cochlear implant system. Step 802 may be performed in any of the ways described herein.

In step 804, the stapedius reflex elicitation and measurement system selects, in accordance with the determined resonant frequency, a probe frequency for use by a middle ear analyzer. Step 804 may be performed in any of the ways described herein.

In step 806, the stapedius reflex elicitation and measurement system directs the middle ear analyzer to generate a measurement probe tone having the selected probe frequency and to use the measurement probe tone to monitor for an occurrence of a stapedius reflex in response to the cochlear implant system applying electrical stimulation by way of one or more electrodes implanted within the patient. Step 806 may be performed in any of the ways described herein.

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media.

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random access memory (“DRAM”), which typically constitutes a main memory. Common forms of computer-readable media include, for example, a disk, hard disk, magnetic tape, any other magnetic medium, a CD-ROM, DVD, any other optical medium, a RAM, a PROM, an EPROM, a FLASH-EEPROM, any other memory chip or cartridge, or any other tangible medium from which a computer can read.

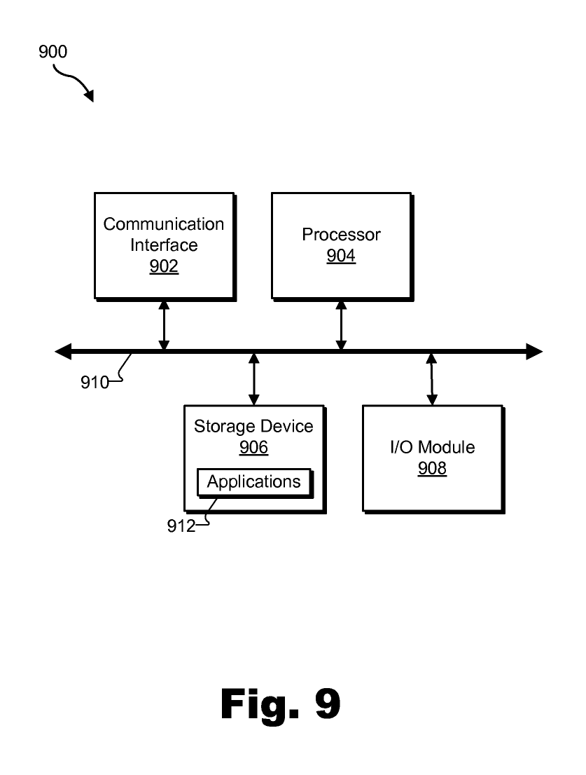

FIG. 9 illustrates an exemplary computing device 900 that may be configured to perform one or more of the processes described herein. As shown in FIG. 9, computing device 900 may include a communication interface 902, a processor 904, a storage device 906, and an input/output (“I/O”) module 908 communicatively connected via a communication infrastructure 910. While an exemplary computing device 900 is shown in FIG. 9, the components illustrated in FIG. 9 are not intended to be limiting. Additional or alternative components may be used in other embodiments. Components of computing device 900 shown in FIG. 9 will now be described in additional detail.

Communication interface 902 may be configured to communicate with one or more computing devices. Examples of communication interface 902 include, without limitation, a wired network interface (such as a network interface card), a wireless network interface (such as a wireless network interface card), a modem, an audio/video connection, and any other suitable interface.

Processor 904 generally represents any type or form of processing unit capable of processing data or interpreting, executing, and/or directing execution of one or more of the instructions, processes, and/or operations described herein. Processor 904 may direct execution of operations in accordance with one or more applications 912 or other computer-executable instructions such as may be stored in storage device 906 or another computer-readable medium.

Storage device 906 may include one or more data storage media, devices, or configurations and may employ any type, form, and combination of data storage media and/or device. For example, storage device 906 may include, but is not limited to, a hard drive, network drive, flash drive, magnetic disc, optical disc, random access memory (“RAM”), dynamic RAM (“DRAM”), other non-volatile and/or volatile data storage units, or a combination or sub-combination thereof. Electronic data, including data described herein, may be temporarily and/or permanently stored in storage device 906. For example, data representative of one or more executable applications 912 configured to direct processor 904 to perform any of the operations described herein may be stored within storage device 906. In some examples, data may be arranged in one or more databases residing within storage device 906.

I/O module 908 may be configured to receive user input and provide user output and may include any hardware, firmware, software, or combination thereof supportive of input and output capabilities. For example, I/O module 908 may include hardware and/or software for capturing user input, including, but not limited to, a keyboard or keypad, a touch screen component (e.g., touch screen display), a receiver (e.g., an RF or infrared receiver), and/or one or more input buttons.

I/O module 908 may include one or more devices for presenting output to a user, including, but not limited to, a graphics engine, a display (e.g., a display screen, one or more output drivers (e.g., display drivers), one or more audio speakers, and one or more audio drivers. In certain embodiments, I/O module 908 is configured to provide graphical data to a display for presentation to a user. The graphical data may be representative of one or more graphical user interfaces and/or any other graphical content as may serve a particular implementation.

In some examples, any of the facilities described herein may be implemented by or within one or more components of computing device 900. For example, one or more applications 912 residing within storage device 906 may be configured to direct processor 904 to perform one or more processes or functions associated with middle ear analyzer 102, interface system 104, cochlear implant system 106, and/or stapedius reflex elicitation and measurement system 700.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A system comprising:
a probe frequency management facility configured to
determine a resonant frequency of a middle ear space of a patient fitted with a cochlear implant system, and
select, in accordance with the determined resonant frequency, a probe frequency for use by a middle ear analyzer; and
a control facility communicatively coupled to the detection probe frequency management facility and configured to direct the middle ear analyzer to generate a measurement probe tone having the selected probe frequency and to use the measurement probe tone to monitor for an occurrence of a stapedius reflex in response to the cochlear implant system applying electrical stimulation by way of one or more electrodes implanted within the patient.

2. The system of claim 1, wherein the probe frequency management facility is configured to determine the resonant frequency by directing the middle ear analyzer to perform a multiple-frequency tympanometry procedure with respect to the patient.

3. The system of claim 1, wherein the probe frequency management facility is configured to determine the resonant frequency in response to input provided by a user.

4. The system of claim 1, wherein the probe frequency management facility is configured to determine the resonant frequency in accordance with a user profile associated with the patient.

5. The system of claim 1, wherein the probe frequency management facility is configured to select the probe frequency in accordance with the determined resonant frequency by selecting a preset frequency option that most closely matches the determined resonant frequency, the preset frequency option included in a plurality of preset frequency options provided by the middle ear analyzer.

6. The system of claim 1, wherein the probe frequency management facility is configured to select the probe frequency in accordance with the determined resonant frequency by designating the determined resonant frequency as the probe frequency.

7. The system of claim 1, wherein the control facility is configured to direct the middle ear analyzer to use the measurement probe tone to monitor for the occurrence of the stapedius reflex by directing the middle ear analyzer to use the measurement probe tone to record a change in acoustic immittance that occurs in response to the cochlear implant system applying the electrical stimulation by way of the one or more electrodes implanted within the patient.

8. The system of claim 1, wherein the control facility is configured to direct the middle ear analyzer to use the measurement probe tone to monitor for the occurrence of the stapedius reflex by directing the middle ear analyzer to apply the measurement probe tone to an ear fitted with the cochlear implant system.

9. The system of claim 1, wherein the control facility is configured to direct the middle ear analyzer to use the measurement probe tone to monitor for the occurrence of the stapedius reflex by directing the middle ear analyzer to apply the measurement probe tone to an ear contralateral to an ear fitted with the cochlear implant system.

10. The system of claim 1, wherein the control facility is further configured to:
identify a current level at which the stapedius reflex occurs; and
use the identified current level to determine one or more most comfortable stimulation levels (“M levels”) associated with the one or more electrodes.

11. An apparatus comprising:
a signal generation facility configured to generate an acoustic signal used to direct a cochlear implant system to apply electrical stimulation having a current level based on a sound level of the acoustic signal by way of one or more electrodes implanted within a patient; and
an analyzer facility communicatively coupled to the signal generation facility and configured to
generate a measurement probe tone having a probe frequency selected in accordance with a resonant frequency of a middle ear space of the patient, and
use the measurement probe tone to monitor for an occurrence of a stapedius reflex in response to the cochlear implant system applying the electrical stimulation by way of the one or more electrodes.

12. The apparatus of claim 12, wherein the signal generation facility is further configured to incrementally increase the sound level of the acoustic signal until the analyzer facility detects the occurrence of the stapedius reflex.

13. A method comprising:
determining, by a stapedius reflex elicitation and measurement system, a resonant frequency of a middle ear space of a patient fitted with a cochlear implant system;
selecting, by the stapedius reflex elicitation and measurement system in accordance with the determined resonant frequency, a probe frequency for use by a middle ear analyzer; and
directing, by the stapedius reflex elicitation and measurement system, the middle ear analyzer to generate a measurement probe tone having the selected probe frequency and to use the measurement probe tone to monitor for an occurrence of a stapedius reflex in response to the cochlear implant system applying electrical stimulation by way of one or more electrodes implanted within the patient.

14. The method of claim 13, wherein the determining of the resonant frequency comprises directing the middle ear analyzer to perform a multiple-frequency tympanometry procedure with respect to the patient.

15. The method of claim 13, wherein the determining of the resonant frequency comprises determining the resonant frequency in response to input provided by a user.

16. The method of claim 13, wherein the determining of the resonant frequency comprises determining the resonant frequency in accordance with a user profile associated with the patient.

17. The method of claim 13, wherein the selecting of the probe frequency in accordance with the determined resonant frequency comprises selecting a preset frequency option that most closely matches the determined resonant frequency, the preset frequency option included in a plurality of preset frequency options provided by the middle ear analyzer.

18. The method of claim 13, wherein the selecting of the probe frequency in accordance with the determined resonant frequency comprises designating the determined resonant frequency as the probe frequency.

19. The method of claim 13, wherein the directing of the middle ear analyzer to use the measurement probe tone to monitor for the occurrence of the stapedius reflex comprises directing the middle ear analyzer to apply the measurement probe tone to an ear associated with the cochlear implant system.

20. The method of claim 13, further comprising:
identifying, by the stapedius reflex elicitation and measurement system, a current level at which the stapedius reflex occurs; and
using, by the stapedius reflex elicitation and measurement system, the identified current level to determine one or more most comfortable stimulation levels (“M levels”) associated with the one or more electrodes.
